(19) United States
(12) Patent Application Publication
(10) Pub. No.: US 2023/0052106 A1
(43) Pub. Date: Feb. 16, 2023

(54) VALVULOTOME

(71) Applicant: Andramed GmbH, Reutlingen (DE)

(72) Inventor: Heinz SCHADE, Reutlingen (DE)

(73) Assignee: Andramed GmbH, Reutlingen (DE)

(21) Appl. No.: 17/794,397

(22) PCT Filed: Jan. 21, 2021
(86) PCT No.: PCT/EP2021/051240

§ 371 Date: Jul. 21, 2022

(30) Foreign Application Priority Data
Jan. 22, 2020 (DE) 10 2020 101 456.2

(51) Int. Cl. A61B 17/22; A61B 17/3207; A61B 90/00

(52) U.S. Cl. CPC A61B 17/22; A61B 17/320725; A61B 17/32075; A61B 90/39; A61B 2017/22097

(57) ABSTRACT

The invention relates to a valvulotome comprising a catheter tube (4) and an expandable basket (5) with cutting elements (7), wherein the catheter tube (4) being designed to accommodate within itself the expandable basket (5) in a non-expanded state, wherein the basket (5) is fixed to the distal end of a wire element (3) extending in the catheter tube (4) and the basket (5) distally comprises an atraumatically configured resilient tip (8).

The invention relates to a valvulotome provided with a catheter tube and an expandable basket with cutting elements, wherein the catheter tube is designed to accommodate within itself the expandable basket which is in an unexpanded state.

When transplanting body-own veins into the coronary arteries region for the purpose of creating bypasses, it is necessary to first remove venous valves from the veins designated for this purpose. Venous valves are known to impair the flow of blood which is objectionable in the case of coronary arteries serving the supply of arterial blood to the myocardium. In this respect, body-own veins meant to create bypasses are as a rule taken from the thigh area of a patient and before grafting must be properly prepared. Provided there is sufficient time, such vein preparation as a rule takes place in the patient's body some time before the bypass operation is performed so that the relevant vessels are given sufficient time for recovery and healing.

To remove venous valves in a patient's body, so-called valvulotomes are used, i.e. endovascular instruments that are inserted into the relevant vessel and “strip” the venous valves with the help of laterally placed cutting blades. Such an intervention is carried out, as a rule, with the help of a catheter inserted into the relevant vessel of the patient at the distal lower leg via the groin or through some other point of access, with a valvulotome, which is known per se, being subsequently transferred through this catheter to the desired application site. Having left the catheter the cutting elements of the valvulotome remove the venous valves which with the aid of a basket-like structure arranged near the tip of the valvulotome are then drawn into the catheter and taken out of the patient's body together with the catheter and valvulotome.

Such a valvulotome has been disclosed, for example, by publication WO 96/33662 A1 and is guided by a cable, equipped with a basket as spacer element as well as cutting tools which are arranged in a cutting head located in front of the basket. An atraumatic tip has been arranged distally.

This valvulotome is brought to the desired location with the help of a catheter but its design lacks a controlling/steering function. In particular, this valvulotome has not been provided with a guidewire serving for control or steering purposes.

Moreover, the basket and the cutting elements of this known valvulotome are arranged separately with the necessity that they must function in concurrence. Its cutting elements are movably arranged in a cutting head and pressed against the vessel wall by means of the basket which consists of spring elements.

From WO 2011/107 249 A1 a valvulotome is known in which the cutting blades are located on an expandable cutting basket, with the cutting basket being the distal end of a tube guided in a catheter. The cutting basket can be accommodated in the catheter in a non-expanded state. The valvulotome is provided with an inner guidewire that serves for control and positioning. A sleeve serves as distal termination of the cutting basket and facilitates expansion.

A disadvantage of this embodiment is the space required by the second tube and the guidewire, with the latter limiting the cutting basket's capacity to accommodate tissue, due to the fact that it is guided through the cutting head. This limits the accommodation capacity of the cutting basket and may lead to the basket not being able to be retracted into the catheter due to adhering tissue remnants.

Furthermore, other valvulotomes have been disclosed that essentially consist of a spring-type basket provided with cutting elements. Such a valvulotome introduced into practice by the company of LeMaitre was also designed for guidance by catheter and had a basket comprising several braces into which cutting elements were integrated. However, these valvulotomes as well were incapable of being guided/steered independently.

Bearing this in mind, it is the object of the present invention to provide a valvulotome capable of being guided independently to the desired application site. Moreover, the valvulotome should be controllable to such an extent that the stripping operation could be controlled by the attending physician and customized to suit the relevant diameter and condition of the patient's vein. In particular, the basket of the valvulotome should be kept free of internal elements in order to provide a large accommodation volume for tissue remnants.

This objective is achieved by providing a valvulotome of the kind first mentioned above, in which the basket is fixed to the distal end of a wire element that extends within the catheter tube and in which the basket distally has an atraumatically designed and resilient tip.

In particular, the cutting basket and the atraumatic tip are made from a tube using a laser cutting technique. This results in the tip and also the cutting basket being hollow.

The valvulotome proposed by the invention is guided in a customary catheter with which it interacts. The catheter is inserted into a patient's vessel in the usual manner and is controlled by the attending physician via the wire element in the usual way. However, the wire element is not a conventional guidewire for the placement of the valvulotome because it distally terminates at the cutting basket of the valvulotome, i.e., it is not guided into and through the cutting basket. It is used exclusively for moving the cutting basket.

The cutting basket is of expandable design, that is, it can be guided within the catheter when it is in non-expanded state and, having left the catheter, expands to assume its expanded state. For this reason, it is fabricated of a spring material, in particular a shape memory material, for example nitinol, which allows the extent of expansion to be predetermined. For implementation and use, the cutting basket is pushed out of the catheter via the wire element.

For this reason, the wire element must have both a certain amount of flexibility and also stiffness. Preferably, the element is therefore of braided wire strand design consisting of fine steel wires that offer sufficient stiffness to push the cutting basket out of the catheter and retract it into the catheter after stripping the venous valves has taken place. Since the wire element does not extend into the basket adequate space is available there to accommodate the cut-off venous valves and withdraw them with the basket into the catheter. This has proven to be problematic with valvulotomes provided with a guidewire extending through the cutting head.

As provided by the invention, the distal end of the cutting basket is furnished with an atraumatic tip that is soft in design to avoid damage to the vessel wall. In this context, a spiral shape has proven to be very advantageous, i.e. the atraumatic tip has at least partially the shape of a helical spring. The spirals of such a helical form can be connected by webs in the manner of a universal joint. Using a laser cutting method when manufacturing the basket with a tip from a tube, such a design can easily be realized. Preferably, the cutting basket and tip are made in one piece by laser cutting, in particular from a suitable tube consisting of shape memory material.

In addition to a partially spiral shape, the atraumatic tip may have several arches distally, which contribute to the soft formation of the tip and its atraumatic character. Preferably, there are three or four arches that are bent inward in the direction of the longitudinal axis of the tip. This configuration serves to control the valvulotome during insertion into a vessel and facilitates positioning.

When transported to the application site the inventive valvulotome with its basket is positioned inside the catheter, i.e. the basket in the catheter assumes an elongated/stretched form. After liberation from the catheter, the basket expands. The atraumatic tip of the cutting basket protrudes from the catheter; this facilitates the control of the valvulotome in the vessel. Accordingly, the control is thus not achieved via a guidewire.

The basket is provided with several struts on which the cutting elements are arranged. Preferably, the basket consists of three struts, each provided with one or two cutting elements. The design with three struts offers the advantage that the basket centers itself in the vessel. With two cutting elements per strut, the cutting elements are located on opposite sides of the strut, but their cutting edges point in the same direction.

The struts of the basket are brought together distally in the atraumatically formed tip. The tip and basket can be glued or welded together, but preferably they are made in one piece from a tube by laser welding, in particular a tube made of a shape memory material.

Preferably, the cutting elements are provided with hook-shaped cutters extending along the plane of the struts. In particular, said hook-shaped cutters are flanked by recesses of the struts so that their cutter faces, especially the cutting curvatures, are kept within the longitudinal extension of the struts. When stripping the venous valves, the valves are then retained in the recess located in front of the cutting edge, clipped by pulling back the valvulotome, and then enter the interior of the basket and are pulled with the basket into the catheter. Two cutting elements per strut will facilitate stripping.

The wire element is movably arranged in the catheter so that the basket can be advanced, rotated and retracted, even several times, in order to free the selected vein section from the venous valves.

The valvulotome provided by the invention can be provided with customary radiopaque marker elements which, in particular, may be arranged in the area of the distal end of the catheter tube.

It is understood that the valvulotome proposed by the present invention can be provided in different sizes, for example with a 5F catheter and a cutting basket cut from a 4F nitinol tube. The wire element consists, for example, of a steel wire strand having a thickness of 0.85 mm.

It shall be understood that the term “distal” as used herein refers to the end of the valvulotome, catheter or wire element facing away from the attending physician, and the term “proximal” refers to the end facing the attending physician.

The invention is explained in more detail by way of the enclosed figures, where

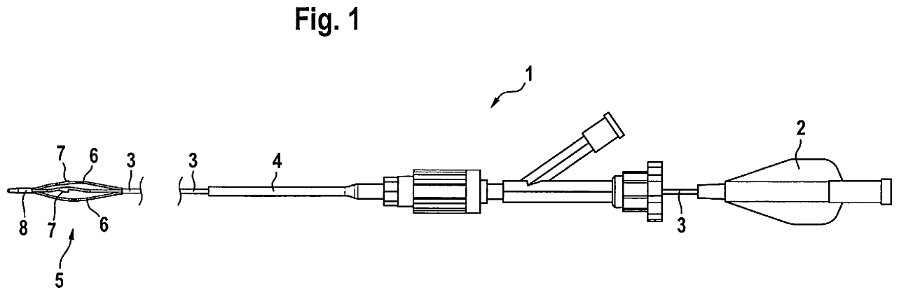

FIG. 1: shows a valvulotome according to the invention with basket in expanded state,

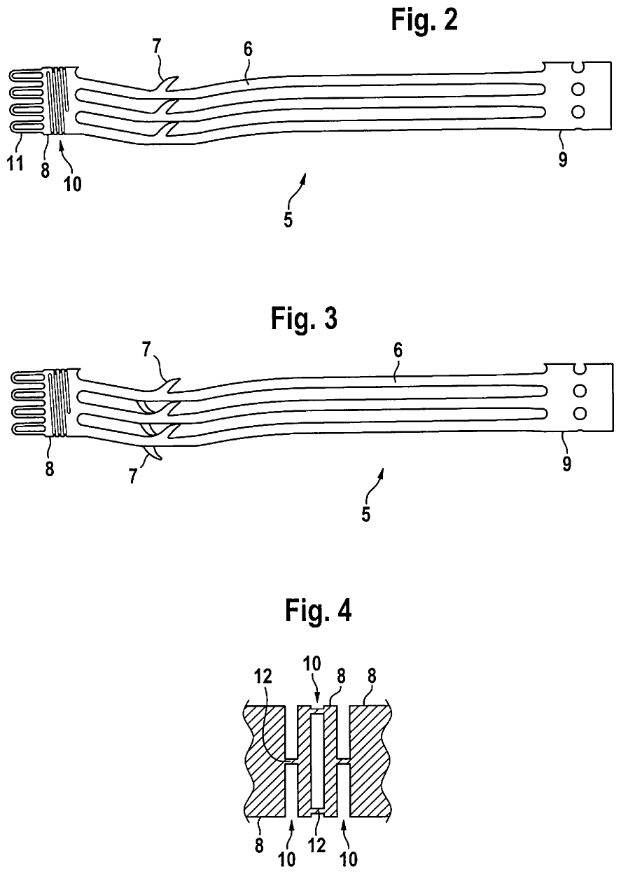

FIG. 2: is a planar view of the basket with 3 cutting elements and tip,

FIG. 3: is a planar view of the basket with 6 cutting elements and tip, and

FIG. 4: shows a universal joint design of the tip.

FIG. 1 shows a valvulotome proposed by the present invention together with the introducer sheath 1, the handle 2 for moving the wire element 3. The valvulotome essentially consists of the catheter tube 4, the wire element 3, the cutting basket 5 and the atraumatically designed soft tip 8. As shown in the figure, the cutting basket 5 is extended from the catheter tube 4, expanded, and ready for insertion into a vein to strip the respective venous valves. It comprises three struts 6, each provided with a cutting element 7. The cutting basket 5 itself is distally provided with an atraumatic and soft tip 8. Cutting basket 5 and tip 8 consist of the same material and are manufactured in one piece.

The wire element 3 is made of a strand of fine steel wires that are braided or twisted together. In this way, the wire element has the flexibility and also the stiffness necessary to move the cutting basket. The wire strand 3 is welded to the proximal end of the cutting basket 5.

The catheter tube 4 is made of conventional material and therefore requires no further explanation. Expediently, the cutting basket 5 is composed of a shape memory material, particularly nitinol, which allows the basket to be imprinted with the desired shape so that it has an elongated form within the catheter tube and assumes the imprinted expanded shape after being liberated from the catheter tube.

FIG. 2 is a planar representation of an inventive variant of a cutting basket 5 with distal tip 8. The basket portion shows three struts 6, with each strut 6 carrying a cutting element 7. The cutting edges 7 are designed to form hook-shaped cutters which are curved in the proximal direction of the struts 6. They are located on the plane of the respective strut. The cutters 7 are flanked by recesses of the struts 6. In the process of stripping the venous valves the valves are retained in the recess located in front of the cutting edge and then clipped by retracting the valvulotome.

Proximal to the cutting basket 5 a tubular segment 9 is arranged, which is triple-perforated and serves to fix the wire strand 3 by welding and/or gluing.

The atraumatically designed tip 8 is located distal to the cutting basket 5, said tip having a spiral incision 10 and a distal end terminating in four arches 11. As mentioned hereinbefore, the tip 8 is formed from a tube. By providing tip 8 with circumferentially extending spiral incision 10, coil spring properties are imparted on the tip and the terminal arches 11 are given a certain amount of elasticity. The terminal arches 11 are bent inward, that is, they are reshaped toward the tube axis, which prevents the tip 8 from contacting the vessel wall.

FIG. 3 illustrates a variant of a cutting basket 5 provided with 2 cutters 7 each. These are located, somewhat offset, on opposite sides of the strut, the cutting edges pointing in proximal direction. Providing two cutting edges per strut as shown in this arrangement increases the “hit rate” when stripping the venous valves.

FIG. 4 shows the universal joint-like design of the incision 10 with webs 12 bridging the incisions. Expediently, two webs 12 are provided per turn, with the webs 12 in adjacent turns being offset from each other by 90°.

1. Valvulotome comprising a catheter tube (4) and an expandable basket (5) with cutting elements (7), wherein the catheter tube (4) being designed to accommodate within itself the expandable basket (5) in a non-expanded state, characterized in that the basket (5) is fixed to the distal end of a wire element (3) extending in the catheter tube (4) and in that the basket (5) distally comprises an atraumatically configured resilient tip (8).

2. Valvulotome according to claim 1, characterized in that the wire element (3) is a braided steel wire strand.

3. Valvulotome according to claim 1, characterized in that the wire element (3) is glued and/or welded to the proximal end of the expandable basket (5).

4. Valvulotome according to claim 1, characterized in that the atraumatically designed tip (8) and the expandable basket (5) are formed in one piece.

5. Valvulotome according to claim 4, characterized in that the atraumatically configured tip (8) and the expandable basket (5) consist of a shape memory material, preferably nitinol.

6. Valvulotome according to claim 5, characterized in that the atraumatically configured tip (8) and the expandable basket (5) are made/cut from a tube.

7. Valvulotome according to claim 6, characterized in that the atraumatically configured tip (8) is provided with a circumferentially running spiral incision (10).

8. Valvulotome according to claim 7, characterized in that incision (10) is provided with webs (12) in the manner of a universal joint.

9. Valvulotome according to claim 1, characterized in that the basket (5) has three struts (6) each provided with one or two cutting elements (7).

10. Valvulotome according to claim 9, characterized in that the struts (6) are brought together distally to form the atraumatically designed tip (8).

11. Valvulotome claim 1, characterized in that the cutting elements (7) have hook-shaped cutters which extend in the plane of the struts (6), with the cutters being flanked by recesses of the struts (6).

12. Valvulotome claim 1, characterized in that the wire element (3) is movably guided in the catheter tube (4).

13. Valvulotome claim 1, characterized in that it comprises a radiopaque marker element in the region of the distal end of the catheter tube (4).
